(19) United States
(12) Patent Application Publication
(10) Pub. No.: US 2024/0041637 A1
(43) Pub. Date: Feb. 8, 2024

(54) UNDERGARMENT FOR COLOSTOMY PATIENTS

(71) Applicant: Nicole Richards, Zimmerman, MN (US)

(72) Inventor: Nicole Richards, Zimmerman, MN (US)

(21) Appl. No.: 18/483,705

(22) Filed: Oct. 10, 2023

Related U.S. Application Data
(63) Continuation-in-part of application No. 17/234,765, filed on Apr. 19, 2021.

(51) Int. Cl. A61F 5/449; A41D 13/12

(52) U.S. Cl. CPC A61F 5/449; A41D 13/1254

(57) ABSTRACT

An undergarment can include a waistband with an internal pocket for placement of a colostomy bag therein. The waistband can completely contain the colostomy bag. A crotch strap can extend from an attached end, at a back of the undergarment, to a removable connection at the front of the undergarment. The user can easily convert the undergarment between underpants, that retain the features of holding the colostomy bag within its waist band, and a simple band for retaining the colostomy bag therein, where the crotch strap can easily fold and store about a back of the waist band.

### CROSS-REFERENCE TO RELATED APPLICATIONS

This application is a continuation-in-part of U.S. patent application Ser. No. 17/234,765, filed Apr. 19, 2021, the contents of which are herein incorporated by reference.

### BACKGROUND OF THE INVENTION

### 1. Field of the Invention

The present invention relates to a garment to be worn by people for supporting an ostomy/colostomy bag attached to an opening called stoma on their abdominal wall following a surgery. The invention, in particular relates to an undergarment with a pocket to hold the ostomy bag and allowing easy wearing and removal of a part of the garment.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Colostomy is a surgical procedure which leaves an opening called stoma in the abdominal wall of the patients. Patients who undergo such a procedure need to wear a colostomy bag attached to the stoma wherein all the bodily waste is discharged. The colostomy bag is either permanent or temporary for some. It changes the lives of people who undergo such surgeries. There is always a need for garments to normalize the lives of patients of colostomy as much as possible. However, all the garments or solutions that exist make it difficult for the patients to even perform normal roles like relieving oneself in the toilet.

Further, one needs easy and fast access to the colostomy bag while on the go for emptying it for the waste that gets stored in there. Usually, it is a cumbersome process to even empty and clean the bag when outside.

The present invention provides a two-piece undergarment system wherein the segment that covers the pelvic area can be removed to relieve oneself without exposing the colostomy bag and the other segment holds and supports the colostomy bag.

### SUMMARY OF THE INVENTION

This Summary is provided to introduce a selection of concepts in a simplified form that are further described below in the Detailed Description of the invention. This Summary is not intended to identify key aspects or essential aspects of the claimed subject matter. Moreover, this Summary is not intended for use as an aid in determining the scope of the claimed subject matter.

The present application discloses an undergarment that supports and hides the colostomy bag attached to the stoma thereby providing discreteness for the patients.

It is an object of this invention to provide a two-piece undergarment system that not only hides the colostomy bag, but also allows easy access to the pelvic area without exposing the colostomy bag.

It is an additional object of the invention to provide a two-piece undergarment system comprising an upper segment and a lower segment of the undergarment, wherein the upper segment of the undergarment acts as a protective belt or waistband around the waist of the user which holds and supports the colostomy bag, whereas the lower segment of the undergarment can be detached from the upper segment of the undergarment, thereby providing easy access to both the colostomy bag and genitals of the patient without removing the entire undergarment.

The upper segment and lower segment of the undergarment are made up of a fabric or combination of fabrics wherein the fabric forming the protective waistband is preferably thick and elastic in order to provide pressure and good support for holding the colostomy bag whereas the fabric in the lower segment is soft, stretchable, flexible and covers the lower pelvic area of the user.

It is another object of this invention to provide an undergarment made up of a fabric that consists of bamboo, cotton, polyester or spandex.

It is a further object of this invention to provide a two-piece undergarment system comprising a pocket on the outside of the fabric of the protective belt or waistband to hold the colostomy bag.

It is still another object of this invention to provide a protective belt or waistband with a pocket consisting of a pouch formed by two layers of fabric of the belt to hold the colostomy bag within it. The feature of the pouch on the waistband which holds the colostomy bag provides a layer of fabric between the skin of the user and the colostomy bag thereby preventing any skin irritation or inflammation which may happen with prolonged use of a colostomy bag attached to a hole or stoma on your body. Furthermore, the pouch holds the bag in its place all the time even when it gets detached from the hole or stoma on the body thereby preventing any embarrassing situations for the user.

The pocket on the protective belt further consists of a vertical opening or slit sewn on the outside fabric of the belt for insertion of the colostomy bag into the pouch, a flap cover on the outside fabric to cover the vertical opening or slit and a snap enclosure on the flap cover to close the flap cover onto the fabric. The features of flap cover and snap enclosure provide extra support to keep the colostomy bag in its place in the pouch and the snap enclosure prevents the bag from coming or falling out of the pouch. Furthermore, a tube hole, in the pouch and on the inside of the protective belt, is provided to be aligned with the stoma or insertion on the body of the patient.

Both upper and lower segments of the undergarment are provided with plurality of snap fasteners in front and back of each segment to attach or detach the lower segment of the undergarment from the upper segment of the undergarment.

Further, the plurality of snap fasteners on each segment of the undergarment consists of male snap fasteners affixed at the bottom of the fabric of the protective waistband and female snap fasteners affixed at the top of the fabric forming the lower segment of the undergarment. The snap fasteners make the fastening and unfastening user friendly wherein the user can easily detach or attach the segments easily and at will anytime, anywhere they desire. In one embodiment, the user can drop down the front lower segment by unfastening the female snaps on the lower segment from the male snaps on the upper segment, while keeping the back lower segment attached, to provide more accessibility to the crotch area of the user without taking off the entire undergarment.

It is yet another object of this invention to provide a level of discreteness for females who have undergone a colostomy procedure.

In a preferred embodiment, a two-piece undergarment system comprises an upper segment as a protective waistband to hold and support the colostomy bag, wherein the protective waistband consists of a thick layer of fabric around the waist of the user, and a pocket located on the exterior of the protective waistband to house the colostomy bag; a lower segment, wherein the lower segment of the garment consists of a layer of fabric covering the pelvic area of the user; at least a few snap fasteners at the front and back of the upper segment and lower segment to attach and detach the lower segment from the upper segment wherein the upper segment consists of male snaps at the bottom of the protective waistband and the lower segment consists of female snaps at the top of the fabric.

Embodiments of the present invention provide an undergarment comprising a waistband having a width sufficient to contain a colostomy bag thereunder; a pocket fabric attached along a lower edge and along side edges thereof, to a back side of the waistband, the pocket fabric configured to be disposed behind at least a portion of the colostomy bag when the undergarment is worn by a user; a crotch fabric fixed to and extending from a lower edge of the waistband a first side of the waistband; a connection end of the crotch fabric operable to removably attach to a first connection region at a second, opposite side of the waistband; and a second connection region at the first side of the waistband operable to attach to the connection end of the crotch fabric.

Embodiments of the present invention provide an undergarment comprising a waistband having a width sufficient to contain a colostomy bag thereunder; a pocket fabric attached along a lower edge and along side edges thereof, to a user-facing side of the waistband, the pocket fabric configured to be disposed behind at least a portion of the colostomy bag when the undergarment is worn by a user; a crotch fabric fixed to and extending from a lower edge of the waistband a rear side of the waistband; a connection end of the crotch fabric operable to removably attach to a first connection region at a front side of the waistband; and a second connection region at the rear side of the waistband operable to attach to the connection end of the crotch fabric, wherein the connection end of the crotch fabric is removably attachable to either the first connection region or the second connection region.

Embodiments of the present invention provide an undergarment comprising a waistband having a width sufficient to contain a colostomy bag thereunder; a pocket fabric attached along a lower edge and along side edges thereof, to a back side of the waistband, the pocket fabric configured to be disposed behind at least a portion of the colostomy bag when the undergarment is worn by a user; a crotch fabric fixed to and extending from a lower edge of the waistband a first side of the waistband; a connection end of the crotch fabric operable to removably attach to a first connection region at a second, opposite side of the waistband; a second connection region at the first side of the waistband operable to attach to the connection end of the crotch fabric; a first connector at the connection end of the crotch fabric; a mating connector at the first connection region, the mating connector forming a removable connection with the first connector; and a second connector at the second connection region, the second connector being a type of connector that is the same as the mating connector, wherein the connection end of the crotch fabric is removably attachable to either the first connection region or the second connection region.

These together with other objects and advantages which will become subsequently apparent reside in the details of the invention as more fully described and claimed, reference being had to the accompanying drawings forming a part thereof, wherein like numerals refer to like parts throughout.

### BRIEF DESCRIPTION OF THE DRAWINGS

Some embodiments of the present invention are illustrated as an example and are not limited by the figures of the accompanying drawings, in which like references may indicate similar elements.

FIG. 1 shows a front view of a preferred embodiment of an undergarment according to the invention;

FIG. 2 shows a right side view of a preferred embodiment of an undergarment according to the invention;

FIG. 3 shows a front view of detached upper and lower segments of an undergarment according to the invention;

FIG. 4: shows a half detached lower segment of an undergarment according to the invention;

FIG. 5 is a front view of an undergarment according to an exemplary embodiment of the present invention;

FIG. 6 is a front view of the undergarment of FIG. 5 with the front connection detached;

FIG. 7 is a rear view of the undergarment of FIG. 5;

FIG. 8 is a front view of the undergarment of FIG. 5, worn by a user, with the front connection attached;

FIG. 9 is a front view of the undergarment of FIG. 5, worn by a user, with the front connection detached;

FIG. 10 is a rear view of the undergarment of FIG. 5, worn by a user, with the front connection detached;

FIG. 11 is a rear view of the undergarment of FIG. 5, worn by a user, with the crotch panel in a storage position;

FIG. 12 is a front view of the undergarment of FIG. 5, worn by a user, with the crotch panel in a storage position;

FIG. 13 is a front view of the undergarment of FIG. 5, worn by a user, showing opening of the pocket prior to insertion of the colostomy bag inside the pocket;

FIG. 14 is a front view of the undergarment of FIG. 5, worn by a user, showing a portion of the colostomy bag positioned inside the pocket;

FIG. 15 is a front view of a two-panel undergarment, according to an exemplary embodiment of the present invention;

FIG. 16 is a side view of the two-panel undergarment of FIG. 15;

FIG. 17 is a back view of the two-panel undergarment of FIG. 15; and

FIG. 18 is another side view of the two-panel undergarment of FIG. 15.

The illustrations in the figures may not necessarily be drawn to scale.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

As is well known to those skilled in the art, many careful considerations and compromises typically must be made when designing for the optimal configuration of a commercial implementation of any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Broadly, embodiments of the present invention provide an undergarment that can include a waistband with an internal pocket for placement of a colostomy bag therein. The waistband can completely contain the colostomy bag. A crotch strap can extend from an attached end, at a back of the undergarment, to a removable connection at the front of the undergarment. The user can easily convert the undergarment between underpants, that retain the features of holding the colostomy bag within its waist band, and a simple band for retaining the colostomy bag therein, where the crotch strap can easily fold and store about a back of the waist band.

FIG. 1 shows a full front view of an undergarment according to the present invention. As shown in FIG. 1, a two-piece undergarment 10 of the present invention comprises an upper segment 11 and a lower segment 12, a protective waistband 13, a pocket 14, a pouch 15, a vertical opening or slit 16, a tube hole 17, a flap cover 18, and a snap enclosure 19.

As illustrated in FIGS. 1, 3 and 4, the upper segment 11 of the undergarment is made up of a thick layer of fabric forms a protective waistband 13 which extends around the waist of the user. The waistband 13 includes a pocket 14 which holds and supports the colostomy bag in its place. The protective waistband 13 formed in the upper segment 11 of the garment 10 is preferably constructed with two layers of fabric to form a cavity or pouch 15 within a pocket 14 to house the colostomy bag inserted through the vertical opening 16 formed on the exterior of the waistband 13. In one embodiment, the whole protective waistband is made up of two layers of fabric. In another embodiment, only the pouch on the waistband is made up of two layers of fabric in order to create a cavity to hold the colostomy bag.

The pocket 14 further includes a flap cover 18 on the exterior of the waistband and the said flap cover 16 covers the vertical opening 16 by means which may include, but not limited to, a snap or hook and loop fastener, such as Velcro®, to form a snap enclosure 19 as shown in FIG. 1. The snap enclosure helps in keeping the bag in the pouch and prevents it from falling or coming out even when it gets detached from the stoma on the abdomen of the user. As illustrated in FIGS. 1, 3 and 4, the pocket 14 also includes a tube hole 17 on one side of the pouch 15 to be aligned with the stoma or hole on the body of the user.

In one embodiment, the pocket on the exterior of the fabric of the upper segment of the undergarment can be on the right or left side of the garment depending upon the location of the stoma.

According to the present invention, the lower segment 12 of the undergarment can be detached from the upper segment 11 of the undergarment 10 whenever the user desires an access to her genitals. The lower segment 12 of the undergarment 10 is made up of a sheet of fabric that extends from bottom of the upper segment in front to the bottom of the upper segment in rear thereby forming leg opening 22, as shown in FIG. 2, on each side and covering the entire pelvic area of the user.

Further, the top strap of the lower segment 12 and bottom strap of the upper segment 11 at the back and front are provided with snap fasteners for easy attachment and detachment of both the segments from each other.

The snap fasteners on upper and lower segments respectively hold the lower segment in its place and also helps in attaching and detaching the lower segment from the upper segment of the undergarment. The snap fasteners located on the upper and lower segment of the undergarment include but not limited to plastic or metal snap buttons, tape, hooks, Velcro. In one embodiment, as illustrated in FIG. 3, the lower segment 12 is provided with, 1 to 5 male snaps 20 in the front and 1 to 8 male snaps, in the back of the upper segment of the undergarment, whereas 1 to 5 female snaps 20 in the front and 1 to 8 female snaps 20 in the back of the lower segment 12 of the undergarment 10 are used to attach and detach the lower segment from the upper segment. In another embodiment, the female snaps are provided on the upper segment and males snaps are provided on the lower segment of the undergarment.

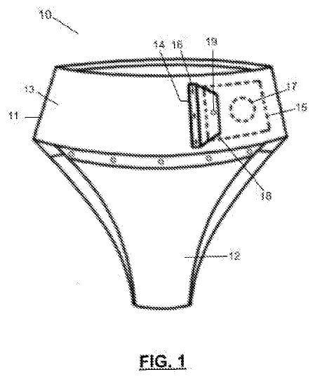

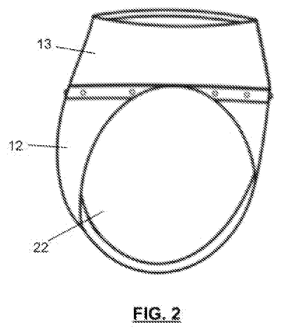

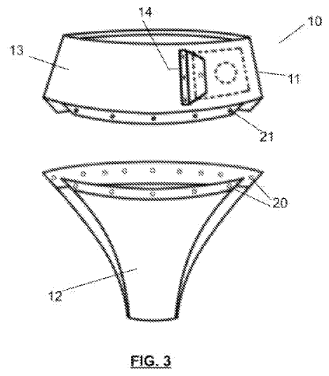

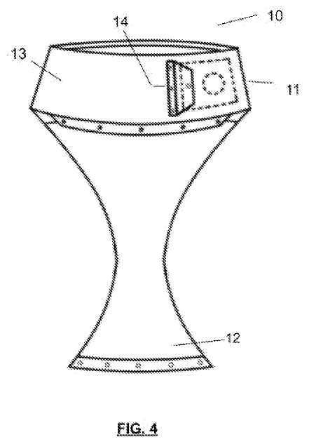

As illustrated in FIGS. 3 and 4, the lower segment 12 of the undergarment of the present invention can be detached and still have the waistband or belt 13 on the body to retain or cover the colostomy bag. In one embodiment, the lower segment 12 of the undergarment 10 of the present invention detaches completely from the upper segment 11 of the undergarment 10, as shown in FIG. 3. The removal of only the lower segment of the undergarment helps the user maintain a normal lifestyle, for e.g., the lower segment can be removed during any medical examination of the pelvic area or during intimate moments without disturbing or worrying about the colostomy bag. In another embodiment, as shown in FIG. 4, the said lower segment 12 can be detached halfway in front, wherein, the back portion of the lower segment 12 remains hooked or snapped at the back with the upper segment 11 of the undergarment 10. The halfway drop down of the lower segment of the undergarment allows the user to relieve oneself without removing the whole undergarment.

The upper segment and lower segment of the undergarment are made up of a fabric or combination of fabrics. According to the present invention, the fabric of the upper and lower segment preferably consists of bamboo, cotton, polyester or spandex. Bamboo provides many benefits for patients who have undergone a colostomy procedure and the benefits include antimicrobial properties, and good wicking properties for the wound whereas spandex material provides elasticity and stretchability to an undergarment.

The undergarment of the present invention can be designed for both male and female users and moreover, the undergarment works for all kinds of drainable and closed colostomy bags. Further, the pouch that houses the colostomy bag can fit bags with variable lengths, shapes and sizes. Furthermore, the undergarment of the present invention can be manufactured for daily wear or can be designed with laces and/or sheer for fancy wear for female users. In addition, the undergarment of the present invention can be made in different varieties including, but not limited to, classic briefs, high-cut briefs, control briefs, hipsters, bikinis, and the like.

The feature of the protective waistband with the pocket and pouch therein, which helps not only in hiding the bag but also supporting and keeping the colostomy bag in its place, and the detachment of the lower segment of the undergarment from the upper segment of the undergarment without removing the whole undergarment, helps in normalizing intimate moments for the patients.

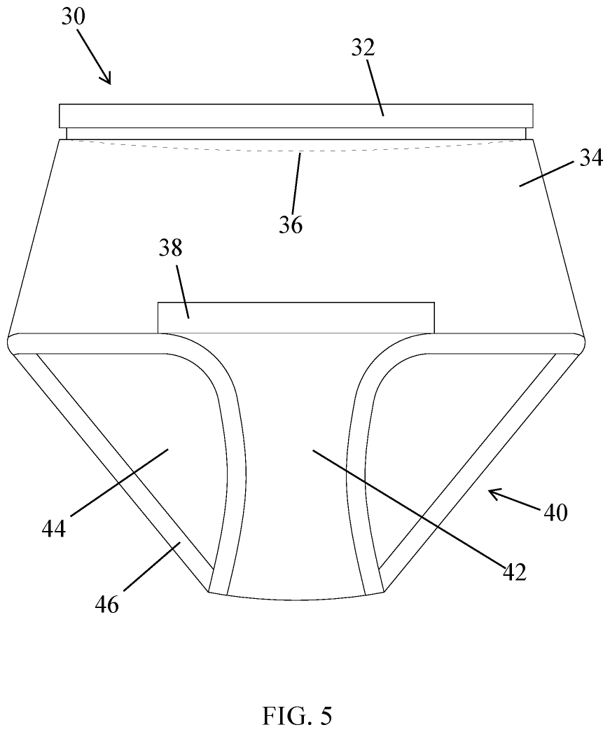

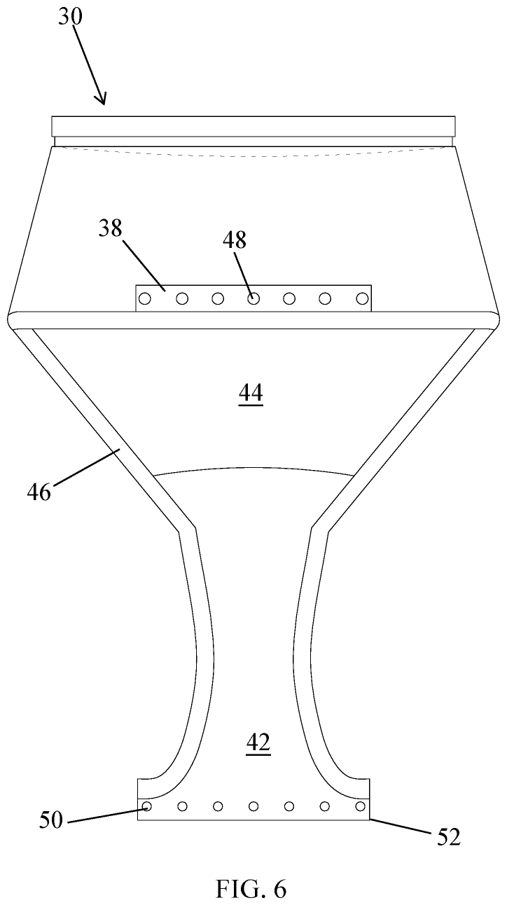

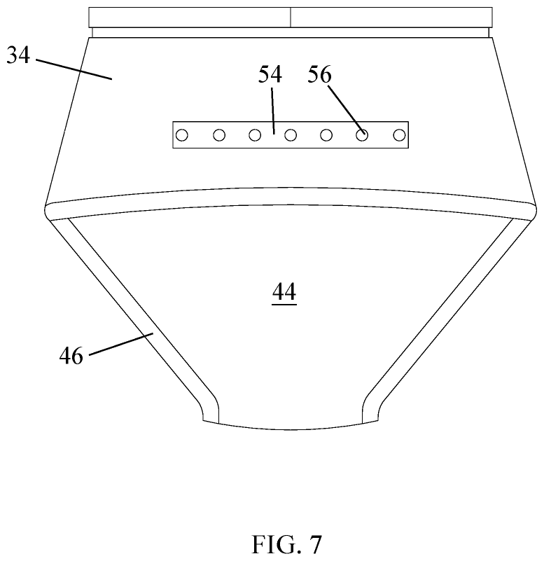

Referring now to FIGS. 5 through 12, an undergarment 30 can include an optional upper elastic band 32 along an upper waist band region thereof. A waist band 34 (which forms the outer surface of the pocket 70) is formed as a loop of fabric. Waist band 34 may be from about three inches to about eight inches in width. Typically, the waist band 34 may be sized with a width sufficient to span a width of a colostomy bag 60 that may be worn by the user.

A crotch fabric 40 may be fixed to a back side of the waist band 34. Typically, the crotch fabric 40 is permanently attached to the waist band 34 by stitching, adhesive or the like. The crotch fabric 40 includes a rear crotch fabric 44, configured to cover a back side of the user when being worn, and a front crotch fabric 42, configured to cover a front side of the user. The front crotch fabric 42 may include a connection end 52 that can connection to a connection area 38 on the front of the waist band 34. When the connection end 52 is attached to the connection area 38, leg exit areas 46 can be formed for the user's legs to be disposed while the undergarment 30 is worn.

In some embodiments, a first connector 48 may be disposed in the connection area 38 and a mating connector 50 may be disposed at the connection end 52 of the front crotch fabric 42. The connector 48 and the mating connector 50 may be any fabric joining connector, such as snaps, hooks and eyes, hood and loop fastener, or the like.

A rear connection area 54 may be disposed at a rear side of the waist band 34. The rear connection area 54 may include a rear connector 56 that is the same type as the mating connector 50, thus permitting the connection end 52 of the front crotch fabric 42 to connect to the rear connection area 54, as described below.

An inner fabric 36 may be disposed behind a front portion of the waist band 34. The inner fabric 36 may be stitched or otherwise joined to the waist band 34 at a lower stitching 64 and at each side thereof with side stitching 62. Typically, the inner fabric 36 may be disposed at the front of the undergarment 30 and can have a width that is from 60 to 100 percent of the width of the waist band 34 and can have a length that is from about 30 to about 70 percent of the length of the waist band 34.

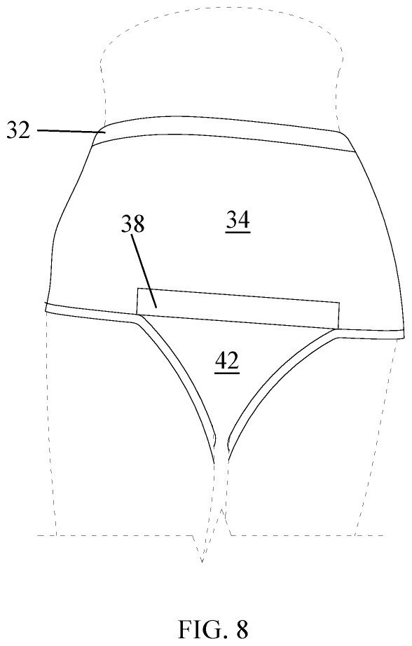

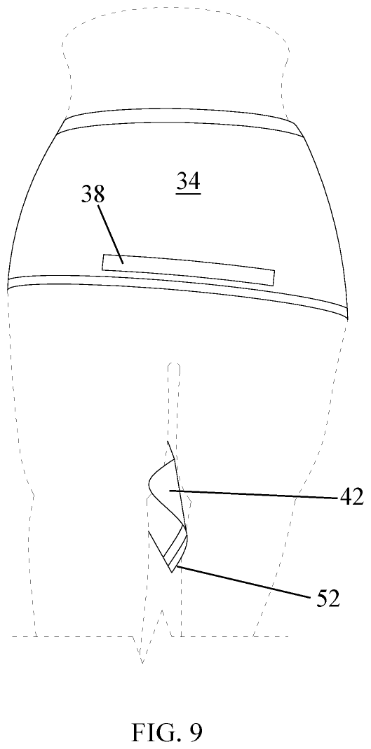

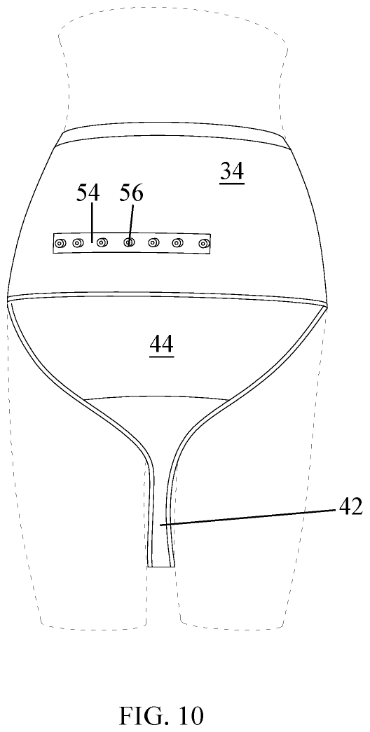

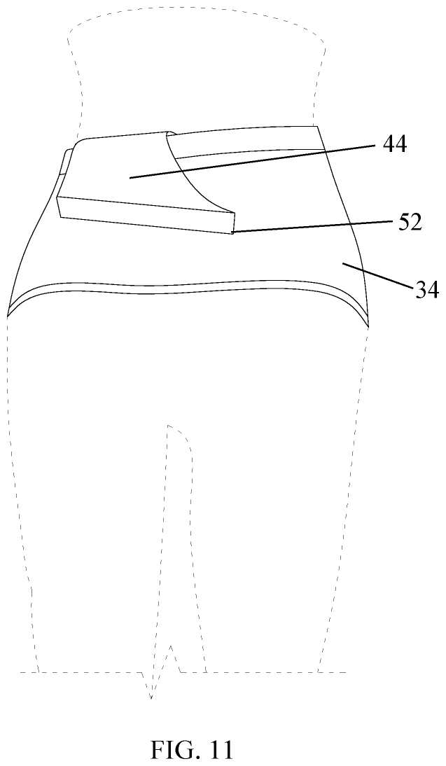

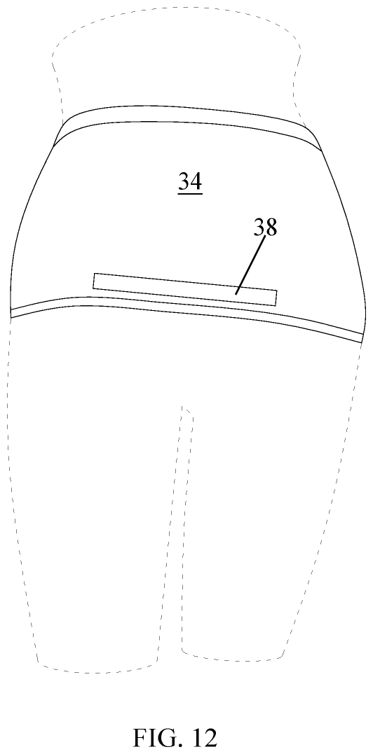

FIGS. 8 through 12 illustrate the undergarment 30 worn by a user and the crotch fabric 40 being moved from a crotch cover position, to a crotch uncover position. In FIG. 8, the undergarment 30 is worn in the user with the crotch fabric 40 extending about the crotch of the user and attached to the front of the waist band 34. In FIG. 9, the user has disconnected the front connection (between connectors 48 and 50, for example, as discussed above). In FIG. 10, a rear view is shown, where the front crotch fabric 42 is hanging down between the user's legs. In FIG. 11, the user can move the crotch fabric 40 under the waist band 34 and optionally attach the connection end 52 of the front crotch fabric 42 to the rear connection area 54, thus preventing movement of the crotch fabric 40. Thus, the undergarment 30 can be transformed from underpants (as shown in FIG. 8) to a colostomy bag band (as shown in FIG. 12), without having to removed and store the crotch fabric.

While the figures show the crotch fabric attached to and extending from a rear side of the waistband and connecting to the front of the waistband, the opposite configuration would also work, where the crotch fabric is fixed to the front of the waistband and can disconnect from a back side thereof. However, in this embodiment, when transforming the undergarment into the band configuration, the movement of the crotch fabric under the waistband would need to avoid interfering with the colostomy bag.

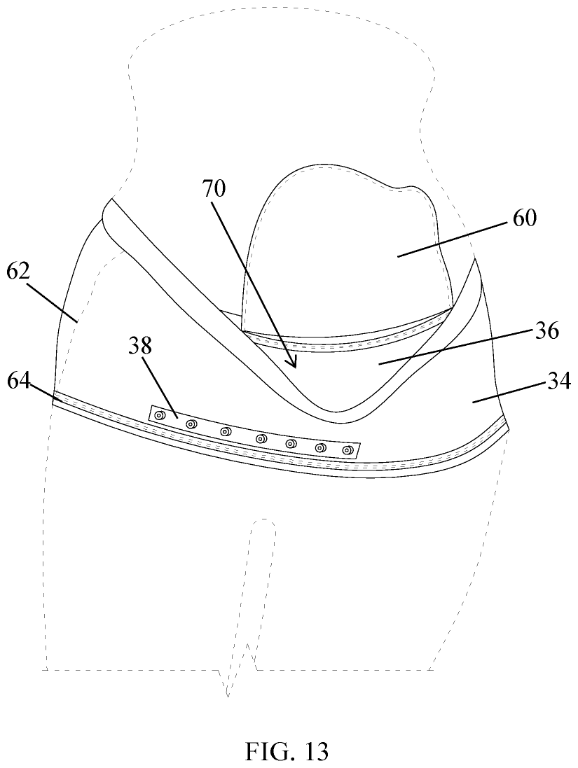

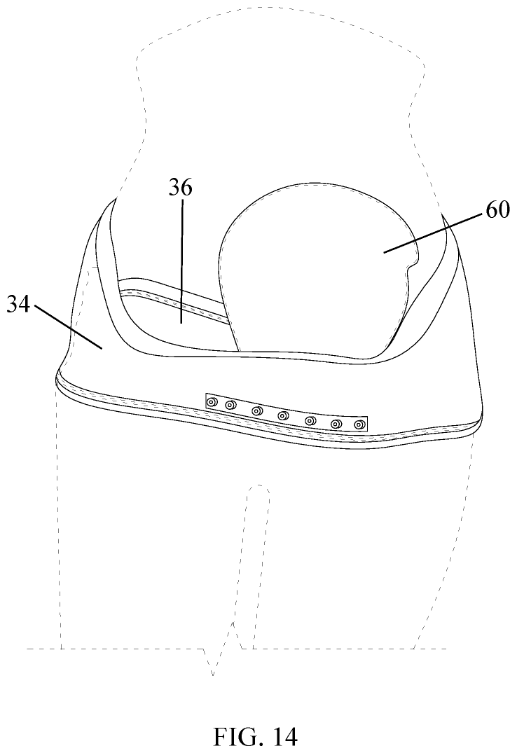

As shown in FIGS. 13 and 14, a colostomy bag 60 on a user can be folded and moved at least partially between the inner pouch fabric 36 and the waist band 34, thus preventing direct contact with a majority of the colostomy bag with the skin of the user. The waist band 34 can completely cover the colostomy bag 60.

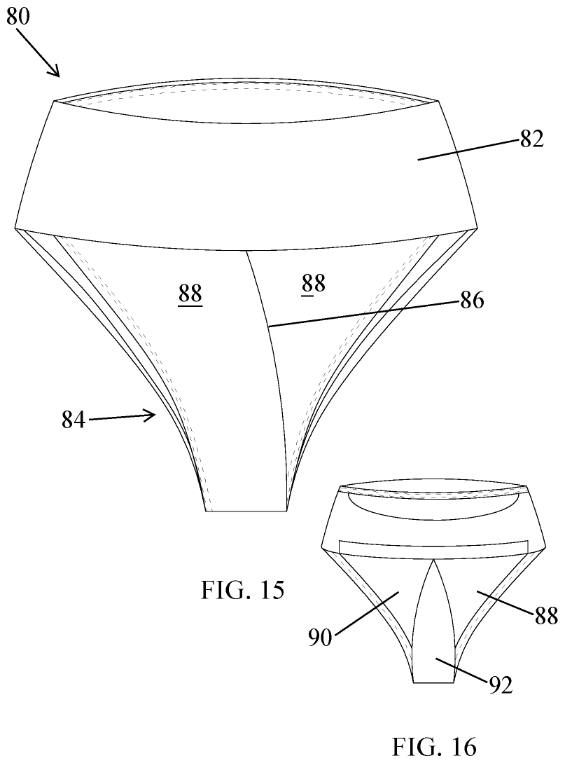

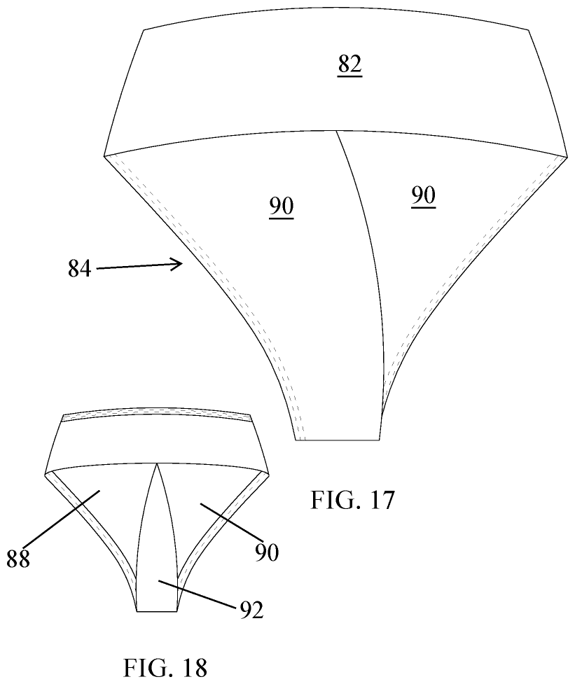

Referring to FIGS. 15 through 18, an alternate embodiment of the undergarment 80 is shown, where, instead of a single piece crotch fabric (such as crotch fabric 40, described above), a double, overlapping fabric crotch area can be formed, where an opening 86 is formed by the overlapping of two pieces of crotch area fabric 84. The undergarment 80 may be identical to that described above, except for the make-up of the crotch fabric. In other words, the undergarment 80 can include the upper waist band 82 and the attachment areas described above. Further, the undergarment 80 can include the pocket formed from a second piece of fabric attached inside of the waist band 82, as described above. Also as in the above embodiment, the embodiment of FIGS. 15 through 18 can also convert between an undergarment and a band by detaching the crotch fabric from the front side, routing the fabric under the inside of the back side of the waist band, and attaching the crotch fabric to the back side of the waist band. The crotch area fabric 84 can include a front crotch fabric 88 and a rear crotch fabric 90, each formed from two pieces of overlapping fabric. Leg openings 90 may be formed on each side of the two pieces of crotch area fabric 84.

In some embodiments, a single length of fabric can form each of the two pieces, from the front crotch fabric 88 to the rear crotch fabric 90. In this embodiment, the two pieces of fabric may fully overlap at a lowermost portion and then separate to still overlap, but not fully, as they move to their respective ends.

The undergarments discussed above can be made from various fabrics, both natural fabrics and synthetic fabrics, or blends thereof. In one embodiment, the undergarments may be formed, at least in part, or entirely, of 95% bamboo and 5% spandex, for example.

While many of the fundamental characteristics and features of the undergarment have been described herein, with reference to particular embodiments thereof, a latitude of modification, various changes and substitutions are intended in the foregoing disclosure, and it should be apparent that in some instances some features of the invention will be employed without a corresponding use of other features without departing from the scope of the invention as set forth.

It should be understood that such substitutions, modifications, and variations may be made by those skilled in the art without departing from the spirit and scope of the invention. Consequently, all such modifications and variations are included within the scope of the invention as defined herein.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Claim elements and steps herein may have been numbered and/or lettered solely as an aid in readability and understanding. Any such numbering and lettering in itself is not intended to and should not be taken to indicate the ordering of elements and/or steps in the claims.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. An undergarment comprising:
a waistband having a width sufficient to contain a colostomy bag thereunder;
a pocket fabric attached along a lower edge and along side edges thereof, to a back side of the waistband, the pocket fabric configured to be disposed behind at least a portion of the colostomy bag when the undergarment is worn by a user;
a crotch fabric fixed to and extending from a lower edge of the waistband a first side of the waistband;
a connection end of the crotch fabric operable to removably attach to a first connection region at a second, opposite side of the waistband; and
a second connection region at the first side of the waistband operable to attach to the connection end of the crotch fabric.

2. The undergarment of claim 1, wherein the first side is a back side of the waistband and the second side is a front side of the waistband.

3. The undergarment of claim 1, further comprising a first connector at the connection end of the crotch fabric.

4. The undergarment of claim 3, further comprising a mating connector at the first connection region, the mating connector forming a removable connection with the first connector.

5. The undergarment of claim 4, further comprising a second connector at the second connection region, the second connector being a type of connector that is the same as the mating connector, wherein the first connector forms a removable connection with the second connector.

6. The undergarment of claim 1, wherein at least a portion of fabric forming the undergarment is 95% bamboo and 5% spandex.

7. The undergarment of claim 1, wherein the crotch fabric is formed from two pieces of crotch fabric overlapping with each other.

8. An undergarment comprising:
a waistband having a width sufficient to contain a colostomy bag thereunder;
a pocket fabric attached along a lower edge and along side edges thereof, to a user-facing side of the waistband, the pocket fabric configured to be disposed behind at least a portion of the colostomy bag when the undergarment is worn by a user;
a crotch fabric fixed to and extending from a lower edge of the waistband a rear side of the waistband;
a connection end of the crotch fabric operable to removably attach to a first connection region at a front side of the waistband; and
a second connection region at the rear side of the waistband operable to attach to the connection end of the crotch fabric, wherein
the connection end of the crotch fabric is removably attachable to either the first connection region or the second connection region.

9. The undergarment of claim 8, further comprising a first connector at the connection end of the crotch fabric.

10. The undergarment of claim 9, further comprising a mating connector at the first connection region, the mating connector forming a removable connection with the first connector.

11. The undergarment of claim 10, further comprising a second connector at the second connection region, the second connector being a type of connector that is the same as the mating connector, wherein the first connector forms a removable connection with the second connector.

12. The undergarment of claim 8, wherein at least a portion of fabric forming the undergarment is 95% bamboo and 5% spandex.

13. The undergarment of claim 8, wherein the crotch fabric is formed from two pieces of crotch fabric overlapping with each other.

14. An undergarment comprising:
a waistband having a width sufficient to contain a colostomy bag thereunder;
a pocket fabric attached along a lower edge and along side edges thereof, to a back side of the waistband, the pocket fabric configured to be disposed behind at least a portion of the colostomy bag when the undergarment is worn by a user;
a crotch fabric fixed to and extending from a lower edge of the waistband a first side of the waistband;
a connection end of the crotch fabric operable to removably attach to a first connection region at a second, opposite side of the waistband;
a second connection region at the first side of the waistband operable to attach to the connection end of the crotch fabric;
a first connector at the connection end of the crotch fabric;
a mating connector at the first connection region, the mating connector forming a removable connection with the first connector; and
a second connector at the second connection region, the second connector being a type of connector that is the same as the mating connector, wherein
the connection end of the crotch fabric is removably attachable to either the first connection region or the second connection region.

15. The undergarment of claim 14, wherein the first side is a back side of the waistband and the second side is a front side of the waistband.

16. The undergarment of claim 14, wherein at least a portion of fabric forming the undergarment is 95% bamboo and 5% spandex.

17. The undergarment of claim 14, wherein the crotch fabric is formed from two pieces of crotch fabric overlapping with each other.
